(19) United States
(12) Patent Application Publication
(10) Pub. No.: US 2014/0296544 A1
(43) Pub. Date: Oct. 2, 2014

(54) CYCLIC PEROXIDE OXIDATION OF AROMATIC COMPOUNDS PRODUCTION AND USE THEREOF

(71) Applicant: Board of Regents, The University of Texas System, Austin, TX (US)

(72) Inventors: Dionicio Siegel, Austin, TX (US); Andrew M. Camelio, Austin, TX (US); Anders Eliasen, Austin, TX (US); Trevor C. Johnson, Austin, TX (US); Abram Axelrod, Hurley, NY (US); Changxia Yuan, Austin, TX (US)

(21) Appl. No.: 13/967,040

(22) Filed: Aug. 14, 2013

Related U.S. Application Data
(60) Provisional application No. 61/805,781, filed on Mar. 27, 2013.

(51) Int. Cl. C07C 29/09; C07C 67/00; C07C 253/30; C07D 311/64; C07C 45/27

(52) U.S. Cl. CPC C07C 29/095; C07D 311/64; C07C 45/27; C07C 253/30; C07C 67/00
USPC 549/408; 568/803; 568/322; 558/426; 568/771; 568/426; 560/75

(57) ABSTRACT

The present invention provides a method for converting an aromatic hydrocarbon to a phenol by providing an aromatic hydrocarbon comprising one or more aromatic C—H bonds and one or more activated C—H bonds in a solvent; adding a phthaloyl peroxide to the solvent; converting the phthaloyl peroxide to a di-radical; contacting the di-radical with the one or more aromatic C—H bonds; oxidizing selectively one of the one or more aromatic C—H bonds in preference to the one or more activated C—H bonds; adding a hydroxyl group to the one of the one or more aromatic C—H bonds to form one or more phenols; and purifying the one or more phenols.

### CROSS-REFERENCE TO RELATED APPLICATIONS

This application claims priority based on U.S. Provisional Application Ser. No. 61/805,781, filed Mar. 27, 2013. The contents of which are incorporated by reference in its entirety.

### STATEMENT OF FEDERALLY FUNDED RESEARCH

This invention was made with government support under Grant Nos. CHE-1059084 and OCI-1053575 both awarded by the National Science Foundation (NSF). The government has certain rights in the invention.

### TECHNICAL FIELD OF THE INVENTION

The present invention relates generally to methods and compositions used in producing a substituted arenes and in particular to oxidative functionalization of aromatic compounds.

### INCORPORATION-BY-REFERENCE OF MATERIALS FILED ON COMPACT DISC

None.

### BACKGROUND OF THE INVENTION

Without limiting the scope of the invention, its background is described in connection with methods and compositions used in producing substituted arenes.

Substituted phenols are broadly useful compounds, functioning as starting materials and end products in all areas of chemical industry. Since the initial discovery of phenol from coal tar advances have been made in the synthetic preparations of this class of compounds which possess a hydroxyl group appended to an aromatic hydrocarbon core. Ideally the synthesis of phenols is achieved through the direct installation of oxygen into an aromatic precursor, which is typically more abundant.

Methods for C—H bond oxidation play a fundamental role in process chemistry, providing functionality that is required in the final target or enables subsequent transformations. The oxidation of aromatic C—H bonds under mild conditions, especially in the context of substituted arenes with diverse functional groups was previously a challenge with the direct hydroxylation of arenes achieved through the use strong Brønsted or Lewis acid to mediated electrophilic aromatic substitution reactions with super-stoichiometric equivalents of oxidants, significantly limiting the scope of the reaction. Classically, the direct hydroxylation of arenes was achieved through the use of strong Brønsted or Lewis acids to mediate electrophilic aromatic substitution reactions with super-stoichiometric equivalents of oxidants, significantly limiting the scope of the reaction. As the products of these reactions are more reactive than the starting materials overoxidation is frequently a competitive process. Transition-metal catalysed C—H oxidation of arenes with or without directing groups has been developed, improving upon the acid-mediated process; however, precious metals are required. Furthermore, terminal alkenes that do not contain activating substituents such as —O-alkyl, aryl (such as phenyl in styrene), cyano, carboxylic ester, or amide near the double bond are difficult to polymerize.

U.S. Pat. No. 8,362,278, entitled, “Methods for the Conversion of a Substituted Furan to a Substituted Pyrrole,” discloses processes for producing substituted pyrrole compounds, such as 2,5-disubstituted pyrroles. Synthetic processes which directly convert substituted furan compounds to substituted pyrrole compounds, via a reaction of the substituted furan compound with ammonia and/or an ammonium salt in the presence of a catalyst.

U.S. Pat. No. 7,022,690, entitled, “Carboxylic acid Substituted Heterocycles, Derivatives Thereof and Methods of Use,” discloses selected carboxylic acid substituted heterocycle compounds that are effective for prophylaxis and treatment of inflammation, tissue degradation, cancer, fibrosis, and related diseases. The subject matter relates to processes for making such compounds as well as to intermediates useful in such processes.

U.S. Pat. No. 8,049,002, entitled, “Processes for Chemical Synthesis of Lipochitooligosaccharides,” discloses processes for the synthesis of lipochitooligosaccharides were developed. A fully acylated oligoglucosamine precursor is prepared and reacted with a glucosamine monomer that has an amine protecting phthaloyl group. With removal of the phthaloyl group, a fatty acid may be added on the terminal glucosamine unit, forming a lipochitooligosaccharide. The processes can be adapted for use on a commercial scale.

### SUMMARY OF THE INVENTION

The present invention provides a method for converting an aromatic hydrocarbon to a phenol by providing one or more aromatic hydrocarbons comprising one or more aromatic C—H bonds in a solvent; adding a phthaloyl peroxide to the solvent; reacting the one or more aromatic hydrocarbons and the phthaloyl peroxide to form one or more phenols, wherein one of the one or more aromatic C—H bonds is selectively oxidized in preference to one or more activated Cₛₚ₃—H bonds; and purifying the one or more phenols. The one or more aromatic hydrocarbons may include one or more functional groups, for example, the functional groups may be selected from alkyl silanes, azides, allenes, nitriles, alkyl boronates, alcohols, halides, and epoxides.

The one or more aromatic hydrocarbons may be single rings, fused ring structures, or ring structures connected by one or more atoms (e.g., one or more polycyclic aromatic hydrocarbons), with the rings individually having 5, 6, 7, 8, 9, 10, 11, or 12 members. The phthaloyl peroxide generates a di-radical that reacts with the aromatic hydrocarbon by a reverse rebound mechanism.

The present invention provides a method of forming (+)-δ-tocopherol by providing dehydroxy-(+)-δ-tocopherol in a solvent; adding a phthaloyl peroxide to the solvent; reacting to form a (+)-δ-tocopherol; regenerating the phthaloyl peroxide; and purifying the (+)-δ-tocopherol.

The present invention provides a method for converting an aromatic hydrocarbon to a phenol by providing one or more aromatic hydrocarbons comprising one or more aromatic C—H bonds in a solvent; adding a phthaloyl peroxide to the solvent; converting the phthaloyl peroxide to a di-radical; contacting the di-radical with the one or more aromatic C—H bonds; oxidizing selectively one of the one or more aromatic C—H bonds in preference to the one or more activated Cₛₚ₃—H bonds; adding a hydroxyl group to the one of the one or more aromatic C—H bonds to form one or more phenols; and purifying the one or more phenols.

The present invention provides a method for the hydroxylation of arenes by 4,5-dichlorophthaloyl peroxide that provides access to phenols starting from the corresponding aryl precursors, replacing C—H bonds with C—OH bonds. The reactivity of 4,5-dichlorophthaloyl peroxide in hexafluoroisopropanol significantly expands the scope of the phthaloyl peroxide-mediated hydroxylation reaction, transforming a variety of arenes with different electronics. In addition, a large number of functional groups are compatible with the present invention including alcohols, diols, amines, esters, aldehydes, ketones, and carboxylic acids. The tolerance of the reaction towards functional groups and the scope of arenes that can be hydroxylated enables streamlined syntheses of diverse phenols.

The present invention provides a method for generating a substituted arene to a phenol by providing one or more aromatic hydrocarbons comprising one or more functional groups in a solvent; adding a halogenated phthaloyl peroxides to the solvent; reacting the one or more aromatic hydrocarbons and the halogenated phthaloyl peroxides to hydroxylate the one or more aromatic hydrocarbons to form one or more substituted arenes comprising a hydroxyl group and the one or more functional groups; and purifying the one or more substituted arenes. The halogenated phthaloyl peroxides may be a poly-chloro phthaloyl peroxide, poly-bromo phthaloyl peroxide, poly-fluoro phthaloyl peroxide or a combination thereof. The one or more functional groups may be selected from an alcohol group, a carbonyl group, an ester group, a methoxy, an ether group, a nitrile. The one or more aromatic hydrocarbons further includes one or more functional groups selected from an alcohol group, a carbonyl group, an ester group, a methoxy, an ether group, or a nitrile. The one or more aromatic hydrocarbons may be a 5, 6, 7, 8, 9, 10, 11, or 12 member ring or fused rings. The method may also include the step of regenerating the halogenated phthaloyl peroxides.

The present invention also provides a method for generating a substituted arene to a phenol (as seen in FIG. 8) by providing one or more aromatic hydrocarbons comprising one or more functional groups in a solvent; adding a halogenated phthaloyl peroxides to the solvent; reacting the one or more aromatic hydrocarbons and the halogenated phthaloyl peroxides to hydroxylate the one or more aromatic hydrocarbons to form one or more substituted arenes comprising a hydroxyl group and the one or more functional groups; and purifying the one or more substituted arenes (as seen in FIG. 8).

### BRIEF DESCRIPTION OF THE DRAWINGS

For a more complete understanding of the features and advantages of the present invention, reference is now made to the detailed description of the invention along with the accompanying figures and in which:

FIG. 1 is a schematic of the diradical activation leading to aryl C—H oxidation through a reverse rebound mechanism or a rebound mechanism.

FIG. 2 is a schematic of the reaction of 1,3,5-trimethylbenzene with phthaloyl peroxide (1) and hydrolysis.

FIGS. 3A-3C are schematics of the phthaloyl peroxide mediated hydroxylation of arenes.

FIGS. 4A-4C are schematics of the hydroxylation of (+)-δ-tocopherol, dehydroabietylamine, and clovanemagnolol derivatives.

FIGS. 5A-5C are schematics of the structures involved in the reverse rebound mechanism.

FIGS. 6A-6B are schematics of the results and computed free energy surfaces for the functionalization of aromatic and benzylic C—H bonds of mesitylene.

FIGS. 7A-7B are schematics of the reaction using phthaloyl peroxide (FIG. 7A) and 4,5-dichlorophthaloyl peroxide (FIG. 7B).

FIGS. 8A-8C are illustrations of the hydroxylation of arenes mediated by 4,5-dichlorophthaloyl peroxide.

FIG. 9 is a schematic of the oxidation reaction of benzene, fluorobenzene, and chlorobenzene.

FIG. 10 is an image of the mixed phthaloyl ester-acids and their respective half-lives.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “arene” denotes aromatic hydrocarbons or aryl hydrocarbon with alternating double and single bonds between carbon or other atoms forming rings. Aromatic compounds and hence aromatic groups may be monocyclic or polycyclic unless otherwise specified. Aromatic compounds include “arenes” (hydrocarbon aromatic compounds) and “heteroarenes,” also termed “hetarenes” (heteroaromatic compounds formally derived from arenes by replacement of one or more methine (—C═) carbon atoms by trivalent or divalent heteroatoms, in such a way as to maintain the continuous pi-electron system characteristic of aromatic systems and a number of out-of-plane pi-electrons corresponding to the Huckel rule (4n+2). The aromatic compounds, arenes, and heteroarenes may be mono- or polycyclic unless otherwise specified. Examples of arenes include, but are not limited to, benzene, naphthalene, and toluene, among others.

As used herein, the term “substituted” may be used to describe an aromatic group wherein any non-hydrogen moiety formally replaces a hydrogen in that group, and is intended to be non-limiting.

As used herein, the term “alkyl silanes” denotes any compound that contains carbon-silicon bonds.

As used herein, the term “azides” denotes any group containing three nitrogens with the formula N₃.

As used herein, the term “allenes” denotes any compound in which one carbon atom has double bonds with each of its two adjacent carbons and include polyenes and dienes.

As used herein, the term “nitriles” denotes any organic compound that has a —C≡N functional group.

As used herein, the term “alkyl boronates” denotes any alkylborate, B(O-Alkyl)₃, and is suitable for use in the reaction described above. Preferred alkylborates include lower alkyl boronates, comprising 1-6 carbon atoms per alkyl group. Examples of preferred alkylboronates include but are not limited to trimethylborate, triethylborate, tributylborate or mixtures thereof.

As used herein, the term “epoxides” denotes a cyclic ether with three ring atoms and can include any compound containing such a ring-shaped organic compound consisting of an oxygen atom bonded to two other atoms, usually of carbon, that are already bonded to each other.

As used herein, the term “Arene” denotes an aromatic hydrocarbon or arene (or sometimes aryl hydrocarbon) is a hydrocarbon with alternating double and single bonds between carbon atoms forming rings. The configuration of six carbon atoms in aromatic compounds is known as a benzene ring, after the simplest possible such hydrocarbon, benzene.

As used herein, the term “Alkyl” denotes optionally substituted straight chain and branched hydrocarbons with at least one hydrogen removed to form a radical group. Alkyl groups include methyl, ethyl, propyl, isopropyl, butyl, isobutyl, t-butyl, 1-methylpropyl, pentyl, isopentyl, sec-pentyl, hexyl, heptyl, octyl, and so on. Alkyl includes cycloalkyl, such as cyclopropyl, cyclobutyl, cyclopentyl, and cyclohexyl. These groups can be optionally substituted with one or more functional groups which are attached commonly to such chains, such as, hydroxyl, bromo, fluoro, chloro, iodo, mercapto or thio, cyano, alkylthio, heterocyclyl, aryl, heteroaryl, carboxyl, carbalkoyl, alkyl, alkenyl, nitro, amino, alkoxyl, amido, and the like to form alkyl groups such as trifluoro methyl, 3-hydroxyhexyl, 2-carboxypropyl, 2-fluoroethyl, carboxymethyl, cyanobutyl and the like. Examples of the substituent group include a halogen atom, hydroxyl cyano, nitro, carboxyl, an alkyl group, an alkenyl group, an aryl group, an alkoxy group, an alkenyloxy group, an aryloxy group, an acyloxy group, an alkoxycarbonyl group, an alkenylcarbonyl group, an aryloxycarbonyl group, sulfamoyl, an alkyl-substituted sulfamoyl group, an alkenyl-substituted sulfamoyl group, an aryl-substituted sulfamoyl group, a sulfonamido group, carbamoyl, an alkyl-substituted carbamoyl group, an alkenyl-substituted carbamoyl group, an aryl-substituted carbamoyl group, an amido group, an alkylthio group, an alkenylthio group, an arylthio group and an acyl group. For example, one, two or three substituents may be selected from halo, trifluoromethyl, trifluoromethoxy, cyano, hydroxy, amino, nitro, trifluoromethylsulphonyl, carboxy, carbamoyl, C1-4alkyl, C2-4alkenyl, C2-4alkynyl, C1-4-alkoxy, C2-4alkenyloxy, C2-4alkynyloxy, C1-4alkylthio, C1-4alkylsulphinyl, C1-4alkylsulphonyl, C1-4 alkylamino, di-C1-4 alkylamino, C1-4-alkoxycarbonyl, N—C 1-4-alkylcarbamoyl, N,N-di-C1-4alkylcarbamoyl, C2-4alkanoyl, C2-4-alkanoylamino, hydroxyC1-4 alkyl, C1-4alkoxyC1-4alkyl, carboxyC1-4alkyl, C1-4-alkoxycarbonylC1-4 alkyl, carbamoylC1-4alkyl, N—C1-4alkylcarbamoylC1-4 alkyl, N,N-di-C 1-4-alkylcarbamoylC1-4alkyl, phenyl, heteroaryl, phenoxy, phenylthio, phenylsulphinyl, phenylsulphonyl, benzyl, benzoyl, heteroaryloxy, heteroarylthio, heteroarylsulphinyl and heteroarylsulphonyl.

As used herein, the term “Alkoxy” denotes an optionally substituted straight chain or branched alkyl group with a terminal oxygen linking the alkyl group to the rest of the molecule. Alkoxy includes methoxy, ethoxy, propoxy, isopropoxy, butoxy, t-butoxy, pentoxy and so on. “Aminoalkyl”, “thioalkyl”, and “sulfonylalkyl” are analogous to alkoxy, replacing the terminal oxygen atom of alkoxy with, respectively, NH (or NR), S, and SO2. Heteroalkyl includes alkoxy, aminoalkyl, thioalkyl, and so on.

The present invention provides a method for oxidative functionalization of alkanes and alkenes. The present invention provides a simple peroxide reagent that alone functions as a selective oxidant for the transformation of arenes to phenols under mild conditions. Although the reaction proceeds through a radical mechanism, aromatic C—H bonds are selectively oxidized in preference to activated Cₛₚ₃—H bonds and a wide array of functional groups are compatible with this reaction. For example, the present invention provides composition and methods for the oxidation of aromatic compounds to the corresponding phenols.

Substituted phenols are broadly useful compounds, functioning as starting materials and end products in all areas of chemical industry. Since the initial discovery of phenol from coal tar, advances have been made in the synthetic preparations of this class of compounds which possess a hydroxyl group appended to an aromatic hydrocarbon core. Ideally the synthesis of phenols is achieved through the direct installation of oxygen into an aromatic precursor, which is typically more abundant. The present invention provides a novel method for the conversion of aromatic hydrocarbons to phenols even when the precursors possess functionality that is incompatible with strongly oxidizing conditions using phthaloyl peroxide, in the absence of other reagents.

The direct hydroxylation of arenes was initially achieved through the use of strong Brønsted or Lewis acids to mediate electrophilic aromatic substitution reactions with super-stoichiometric equivalents of oxidants, significantly limiting the scope of the reaction. As the products of these reactions are more reactive than the starting materials, overoxidation is frequently a competitive process. Transition-metal catalysed C—H oxidation of arenes with or without directing groups has been developed, improving upon the acid-mediated process; however, precious metals are required. The present invention provides phthaloyl peroxide that functions as a selective oxidant for the transformation of arenes to phenols under mild conditions. This method provides a cost effective reaction that enables the installation of oxygen into aromatic compounds. Methods for this process typically utilize multiple steps and metal catalysts. The present invention simplifies and makes the process more economically advantageous. In addition, the present invention provides a process that is more cost effective and can be performed on a more diverse array of starting materials than current methods permit. This will enable the manufacture of existing value added chemicals as well as the bulk production of chemicals that are not currently accessible using existing transformations. The method currently uses a reagent that cannot be heated above 110° C. The preparation of new derivatives with better thermal stability could be achieved if needed. The present invention provides methods that can extend to the oxidative functionalization of alkanes and alkenes.

The present invention provides the use of phthaloyl peroxide, in the absence of other reagents, for the conversion of aromatic hydrocarbons to phenols even when the precursors possess functionality that is incompatible with strongly oxidizing conditions. The reaction is shown to proceed through a “reverse rebound” mechanism as opposed to the classical rebound mechanism, providing insight into the unique aryl selectivity of the chemical transformation.

Methods for C—H bond oxidation play a fundamental role in synthetic chemistry, providing functionality that is required in the final target or enabling subsequent transformations. Several approaches to aliphatic C—H oxidation have been utilized, dramatically simplifying complex-molecule synthesis. However, the selective oxidation of aromatic C—H bonds under mild conditions, especially in the context of substituted arenes with diverse functional groups, remains a challenge. The direct hydroxylation of arenes was initially achieved through the use of strong Brønsted or Lewis acids to mediate electrophilic aromatic substitution reactions with super-stoichiometric equivalents of oxidants, significantly limiting the scope of the reaction. As the products of these reactions are more reactive than the starting materials overoxidation is frequently a competitive process. Transition-metal catalyzed C—H oxidation of arenes with or without directing groups has been developed, improving upon the acid mediated process; however, precious metals are required. The present invention demonstrates that phthaloyl peroxide functions as a selective oxidant for the transformation of arenes to phenols under mild conditions. Although the reaction proceeds through a radical mechanism, aromatic C—H bonds are selectively oxidized in preference to activated Cₛₚ₃—H bonds.

The present invention provides a wide array of substituted functional groups which are compatible with this reaction, and therefore this method is well suited for late-stage transformations of advanced synthetic intermediates. Density functional theory (DFT) calculations indicate that this transformation proceeds through a novel addition-abstraction mechanism, a kind of “reverse rebound” mechanism as contrasted to the common oxygen rebound mechanism observed for metal-oxo oxidants. These calculations also identify the origins of the observed aryl selectivity.

Phthaloyl peroxide (1) is a unique molecule as homolysis of the peroxide bond generates:

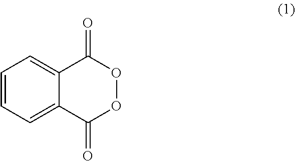 [SMILES: O=c1ooc(=O)c2ccccc12]

a compound possessing two radicals that readily recombine, regenerating the parent peroxide. Although phthaloyl peroxide was first described in the 1950's, there have been minimal studies examining its reactivity. The diradical intermediate generated through homolysis provides opportunities for the development of new reactions, in particular reactions that lead to the oxidative functionalization of C—H bonds.

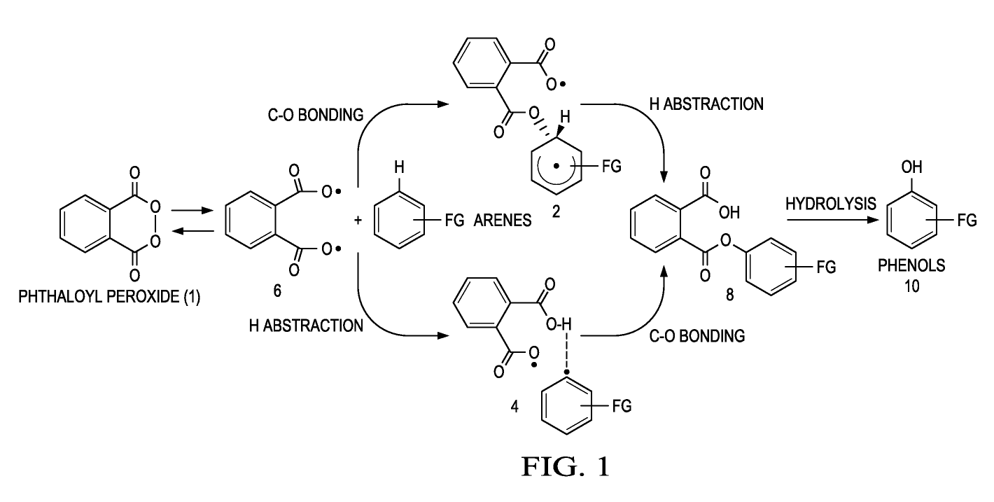

FIG. 1 is a schematic of the diradical activation leading to aryl C—H oxidation through a reverse rebound mechanism or a rebound mechanism. Two possible modes for the reaction of phthaloyl peroxide (1) with arenes; a reverse rebound mechanism proceeding through a cyclohexadienyl radical 4 and a rebound mechanism proceeding through an aryl radical 2. The reaction of arenes with phthaloyl peroxide was envisioned to proceed: first phthaloyl peroxide (1) undergoes a unimolecular reaction to generate diradical 6, the combination of one benzoyloxy radical with an arene generates a cyclohexadienyl radical intermediate 4 (C—O bonding), and lastly the remaining benzoyloxy radical abstracts hydrogen adjacent to the cyclohexadienyl radical (H abstraction) to give phthaloyl ester 8 to yield the final phenol product 10 after hydrolysis. This is a “reverse rebound” mechanism to contrast with metal-oxo or dioxirane oxidations involving hydrogen abstraction followed by C—O bonding through oxygen rebound. Here the normal rebound mechanism involving complex 2 is also possible for the generation of ester phthaloyl ester 8. To test the reactivity of phthaloyl peroxide (1) and to evaluate the selectivity of arene versus Cₛₚ₃—H functionalization initial reactions were conducted using 1,3,5-trimethylbenzene.

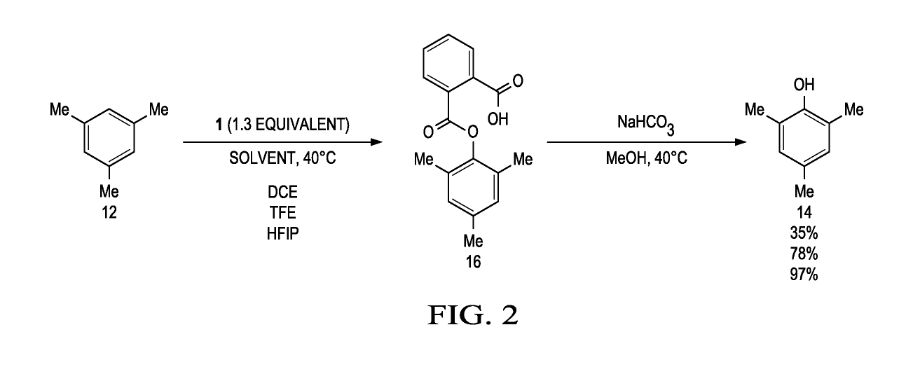

FIG. 2 is a schematic of the reaction of 1,3,5-trimethylbenzene 12 with phthaloyl peroxide (1) and hydrolysis. Abbreviated optimization of the aromatic hydroxylation reaction and DCE, dichloroethane. TFE, trifluoroethanol. HFIP, hexafluoroisopropanol were used in the reaction to produce the final 2,4,6-trimethylphenol 14 through the intermediate 16.

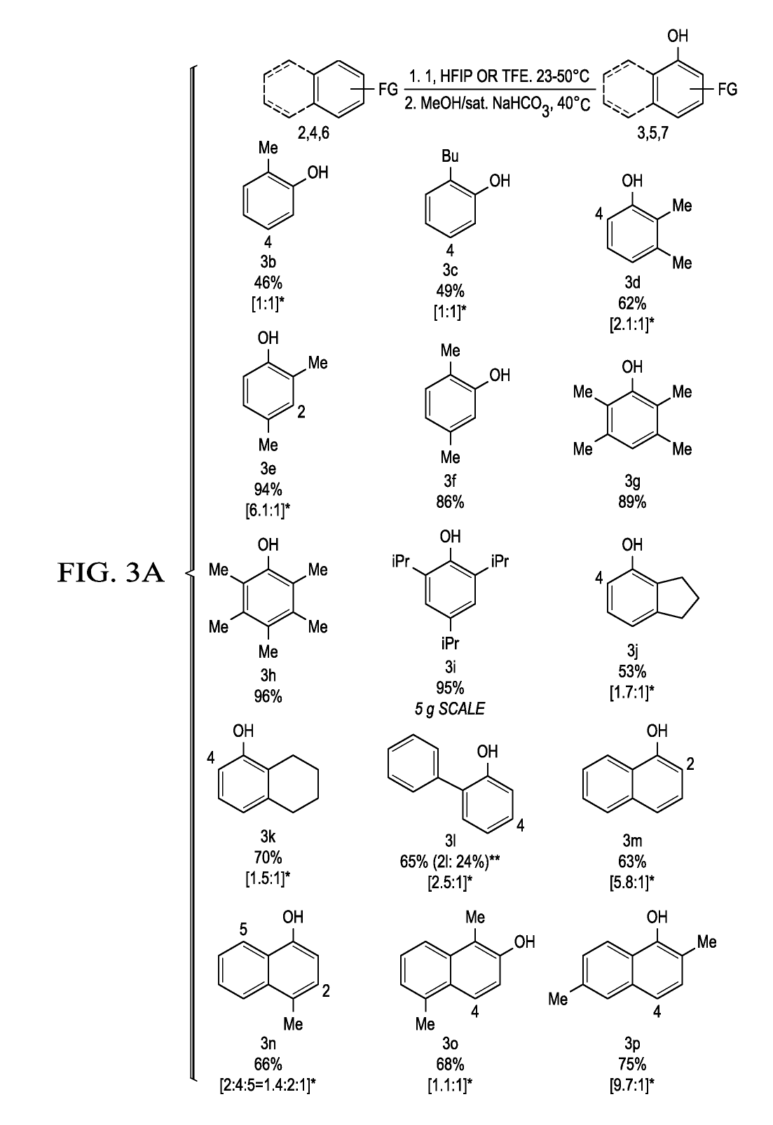

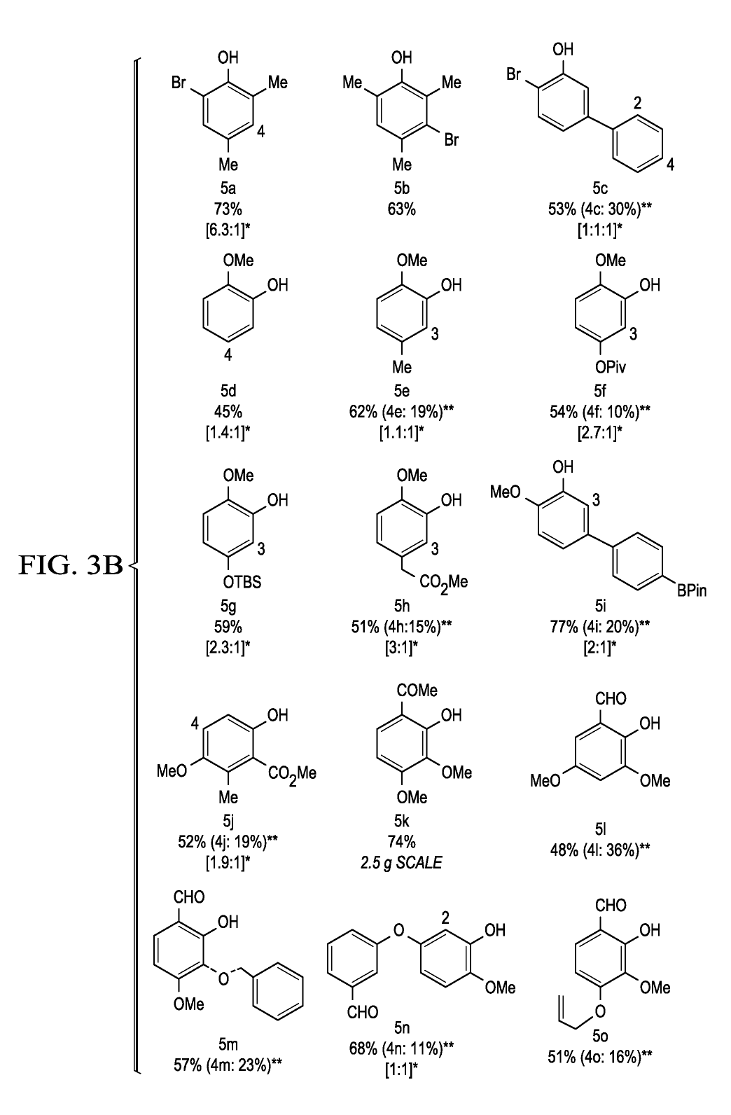

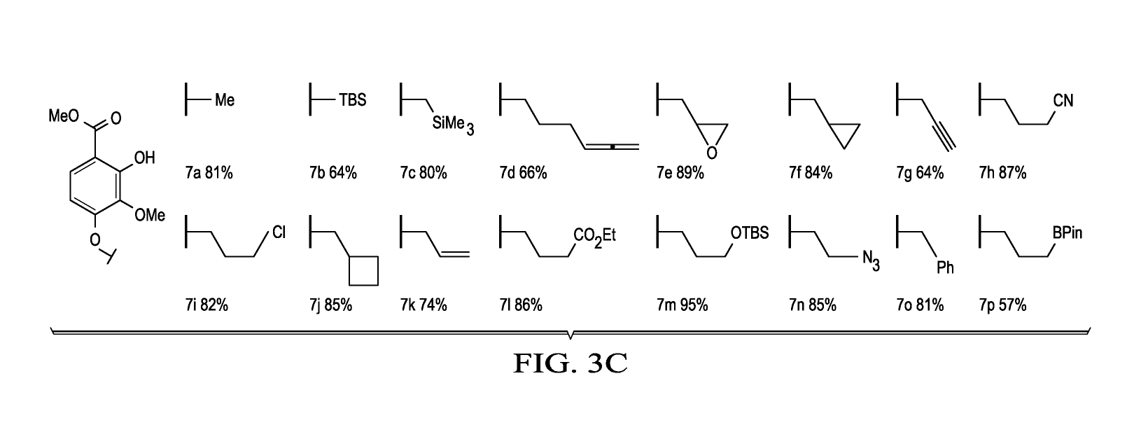

FIG. 3 is a schematic of the phthaloyl peroxide (1) mediated hydroxylation of arenes. FIG. 3A is a table of the hydroxylation of simple and polycyclic arenes. FIG. 3B is a table of the hydroxylation of functionalized arenes. FIG. 3C is a table of the functional group compatibility test: Hydroxylation of methyl vanillate derivatives. Isolated yields are indicated below each entry. iPr═CH(CH₃)₂. Bpin, pinacol boronate. HFIP, hexafluoroisopropanol. TFE, trifluoroethanol. FG, functional groups were used. *The minor regioisomeric position is labeled with the respective carbon atom number. **The yield in parentheses refers to the starting material recovered.

Preliminary attempts generated 2,4,6-trimethylphenol 14 in 35% yield without evidence of benzylic oxidation products or overoxidation. Optimization of the reaction conditions was achieved through the use of trifluoroethanol (TFE) or hexafluoroisopropanol (HFIP) as solvent increasing the yield of the reaction to 78% and 97%, respectively. After identifying the optimal conditions, the hydroxylation of a broad range of arenes was examined. For simple and polycyclic arenes the functionalization proceeds smoothly at 23-50° C. in moderate to excellent yields (46-96%). The transformation can be performed on the multi-gram scale with no need for the exclusion of oxygen and water. In the case of substrates with different aromatic C—H bonds, the oxidation occurs with selectivity that at first approximation parallels Friedel-Crafts reactivity, although the ratio of ortho and para products in many instances favor the ortho products. In all of the substrates examined, including 1,3,5-triisopropylbenzene 2i, the aromatic C—H bond reacts in preference to the benzylic C—H bond. The products in FIGS. 3B and 3C illustrate the range of functional groups that are tolerated in the aromatic C—H oxidation transformation.

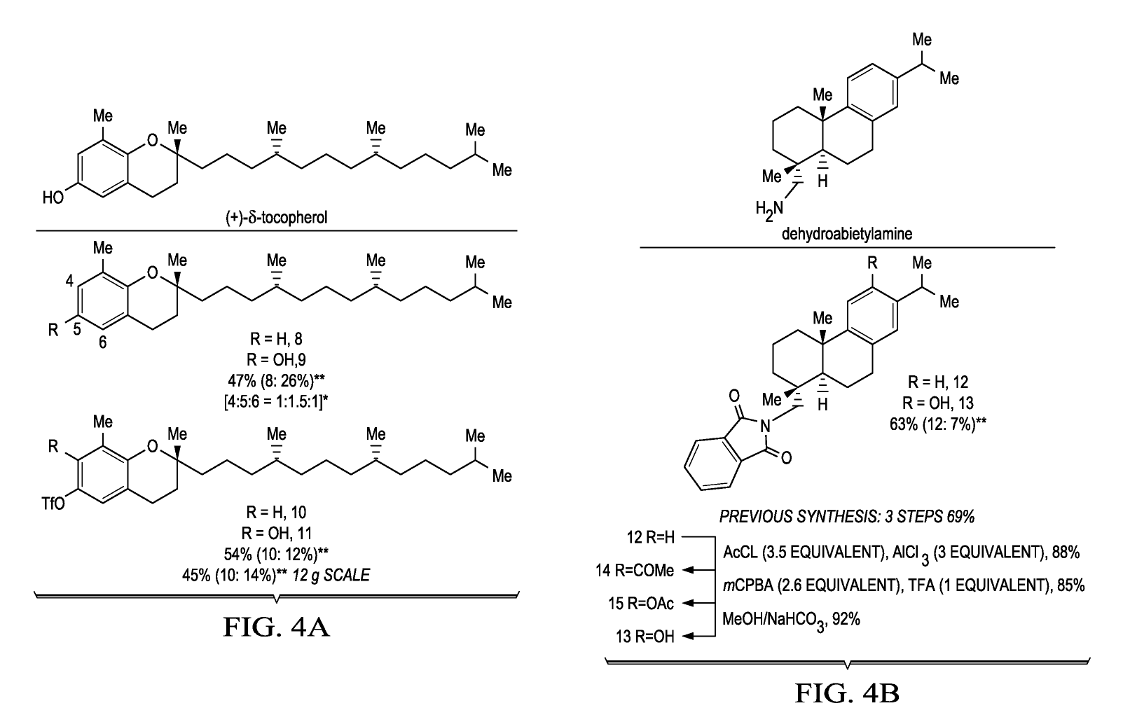

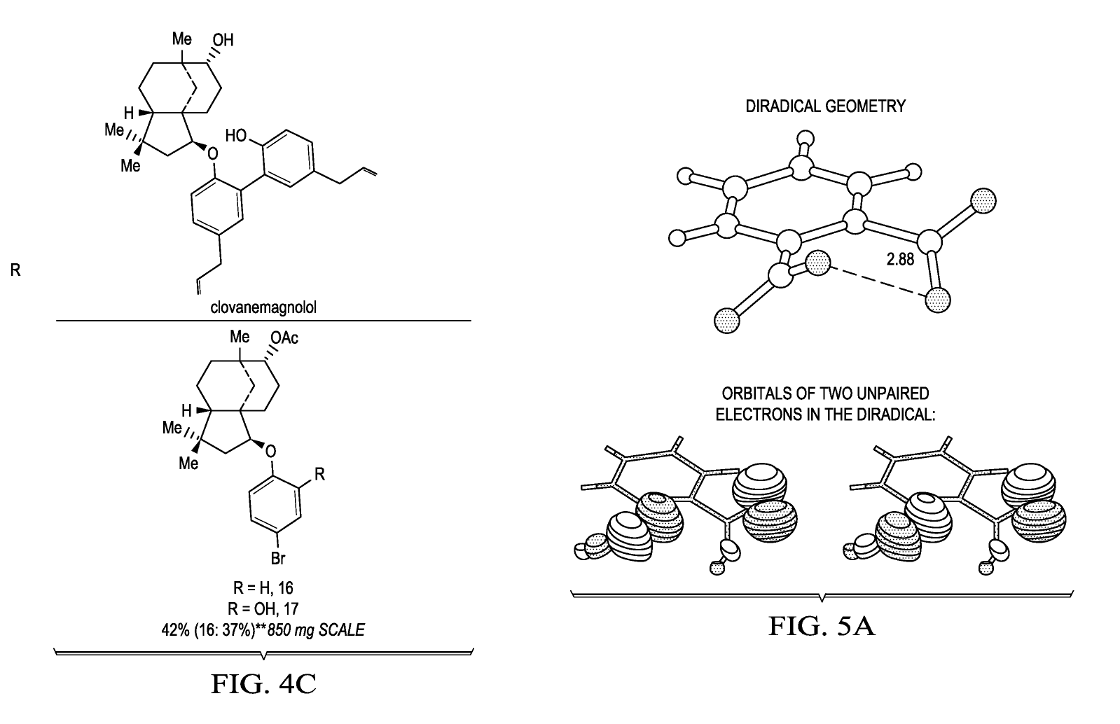

FIGS. 4A-4C are schematics of the hydroxylation of (+)-δ-tocopherol FIG. 4A, dehydroabietylamine FIG. 4B, and clovanemagnolol FIG. 4C derivatives. FIG. 4A illustrates the preparation of (+)-δ-tocopherol and its derivatives. FIG. 4B illustrates the comparison of the synthesis of dehydroabietylamine derivatives using a standard Friedel-Crafts/Baeyer-Villiger sequence. FIG. 4C illustrates the functionalization of the clovanemagnolol precursor. Isolated yields are indicated below each entry. Tf, trifluoromethanesulfonate. Ac, acetyl were used. *The minor regioisomeric position is labeled with the respective carbon atom number. **The yield in parentheses refers to the starting material recovered. Aryl bromides 4a-c were compatible under the reaction conditions. Anisole derivatives 4d-o also gave the expected products following reaction with phthaloyl peroxide (1). Hydroxylation of biaryl 4i was selective for the more electron rich aryl ring and was accomplished without competitive oxidation of the boronate ester. Aryl ketone 4k and aldehydes 4l-o also underwent hydroxylation, whereas the use of other oxidants presents a challenge due to competing Baeyer-Villiger oxidations. The reactions of 4m and 4o cleanly provided products as well deviating from patterns seen with Friedel-Crafts reactivity. The successful hydroxylation of these substrates led to the systematic examination of functional groups that are inert under the reaction conditions through the use of a series of functionalized vanillate derivatives as seen in FIG. 3C. The reaction conditions were compatible with a wide range of functional groups including; alkyl silanes, azides, allenes, nitriles, alkyl boronates, alcohols, halides, and epoxides. Interestingly, the allyl ether 6k reacted selectively at the arene despite the known reaction of phthaloyl peroxide with alkenes and the highly activated methylene of the allylic ether.

This transformation is amenable to late-stage oxidative functionalization of synthetic intermediates en route to complex molecules for biological evaluation. One example is the natural product (+)-δ-tocopherol, which decreases the incidence of prostate cancer as demonstrated in a clinical trial. The oxidation of dehydroxy-(+)-δ-tocopherol with phthaloyl peroxide delivered tocopherols in 47% yield (as seen in FIG. 4A). Treatment of triflate at 23° C. with peroxide (1) produced phenol 11 in 54% yield (this reaction was also conducted on the 12 g scale in 45% yield). With the triflate functioning as an excellent synthetic handle for coupling reactions, the study of the (+)-δ-tocopherol derivatives can be easily pursued. Dehydroabietylamine derivatives have shown important biological effects including the reduction of inflammatory responses, potentially functioning as a phospholipase-A2 inhibitor. The hydroxylation of the dehydroabietylamine derivative 12 with phthaloyl peroxide (1) provided phenol 13 in 63% yield, comparing well with the existing method for introducing phenolic functionality (as seen in FIG. 4B). A direct comparison illustrates how the phthaloyl peroxide process circumvents Friedel-Crafts/Baeyer Villiger sequences, improving upon the step economy. A derivative of the natural product clovanemagnolol was selected due to its importance in regenerative science. Following the literature synthesis bromide 16 was prepared and subjected to the phthaloyl peroxide (1) mediated oxidation to give the hydroxylated product 17 cleanly (as seen in FIG. 4C). On the basis of DFT calculations, this metal-free aromatic C—H oxidation is most likely to occur through a reverse rebound diradical mechanism.

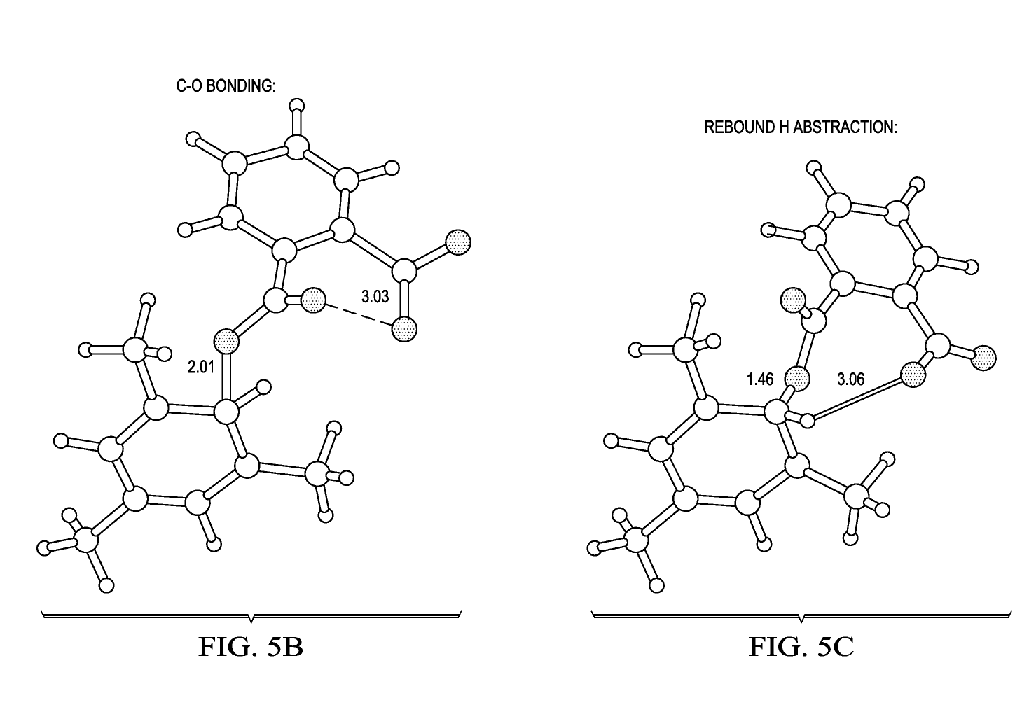

FIGS. 5A-5C are images of the structures involved in the reverse rebound mechanism. The structures include the diradical geometry (FIG. 5A), carbon-oxygen bonding (FIG. 5B), and rebound hydrogen abstraction (FIG. 5C) with the distances given in Å.

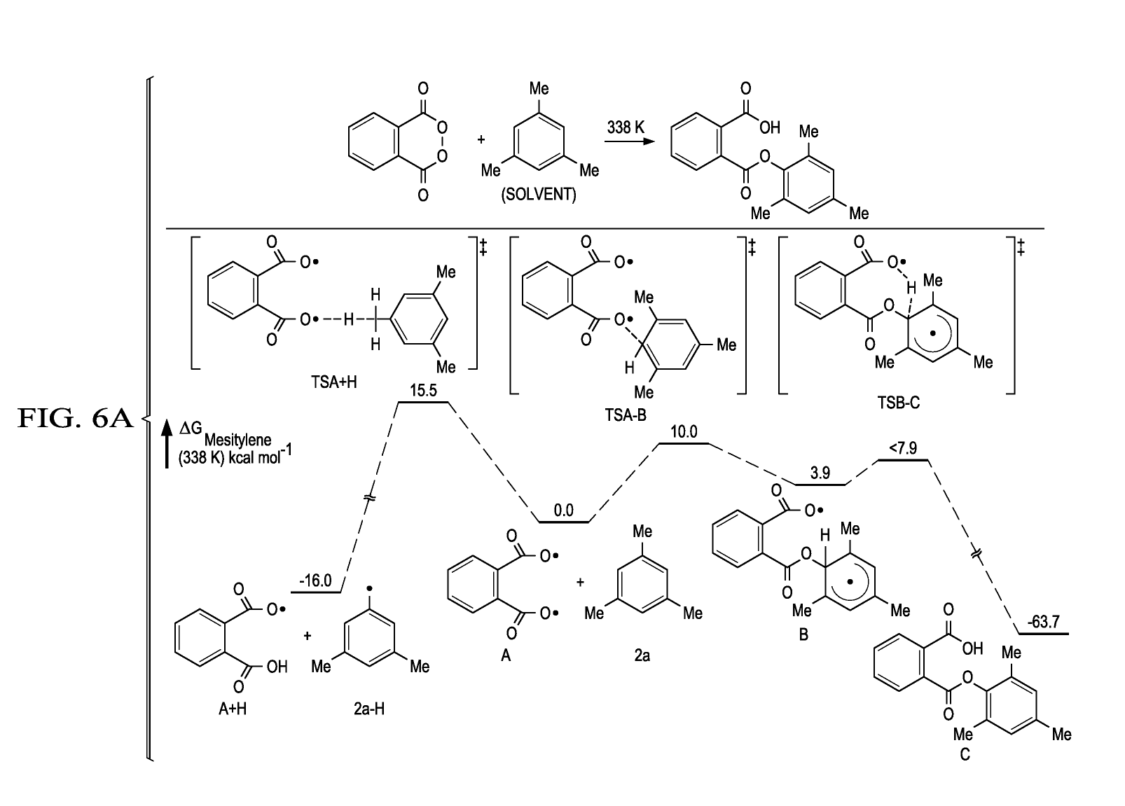

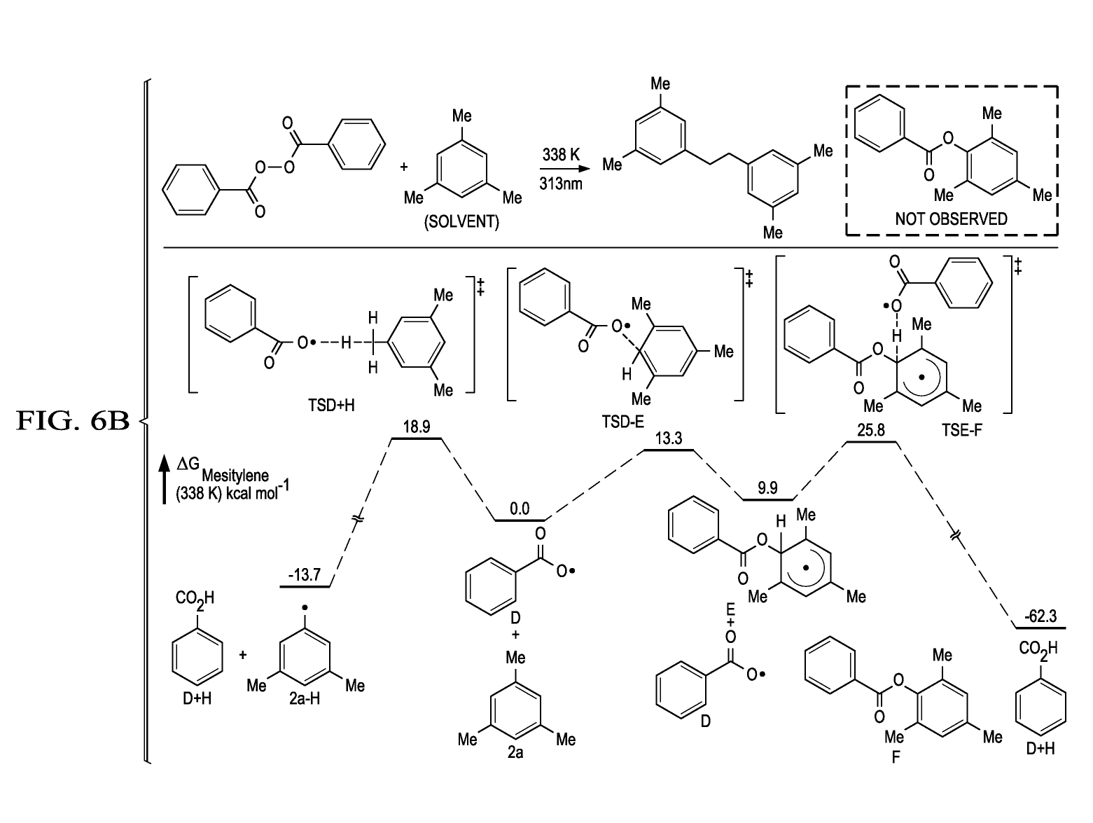

FIGS. 6A and 6B illustrate the schematic and (U)B3LYP/6-31+G(d) computed free energy surfaces for the functionalization of aromatic and benzylic C—H bonds of mesitylene. FIG. 6A illustrates the schematic and (U)B3LYP/6-31+G(d) computed free energy surfaces for diradical A generated from the thermal decomposition of phthaloyl peroxide. FIG. 6B illustrates the schematic and (U)B3LYP/6-31+G(d) computed free energy surfaces for benzoyloxy radical D generated from benzoyl peroxide under irradiation with 313 nm light. To understand the aryl-specific character of this diradical mediated process, we investigated and compared the reaction of the aromatic and benzylic C—H bonds of mesitylene by employing diradical A and benzoyloxy radical D using quantum mechanical calculations. As illustrated in FIG. 6A, the combination of one radical in A with the aromatic ring of mesitylene requires a free energy of 10.0 kcal mol-1. The subsequent intramolecular hydrogen transfer in intermediate B is very facile with a barrier of less than 4 kcal mol-1. Therefore, the radical addition step is rate-determining in the diradical mediated aromatic C—H oxidation. The competing aliphatic C—H functionalization is achieved through the direct hydrogen abstraction to form benzylic radical 2a-H. The required free energy for this process is 5.5 kcal mol-1 higher than that for the aromatic C—H functionalization, accounting for the exclusive aryl selectivity. Interestingly, it was reported that the reaction between benzoyloxy radical D and mesitylene (2a) only gave the aliphatic C—H functionalized product under similar conditions as seen in FIG. 6B. The computed activation free energy of the benzylic hydrogen abstraction by benzoyloxy radical D is 18.9 kcal mol-1. In the two-step aromatic C—H functionalization, the intermolecular hydrogen transfer from radical intermediate E to D becomes rate-determining with a much higher overall barrier of 25.8 kcal mol-1 (as seen in FIG. 6B). This is in agreement with the experimental fact that benzoyloxy radical mediated aromatic C—H oxidation is not observed. For the functionalization of the aliphatic C—H bond of mesitylene, both diradical A and radical D undergo a bimolecular hydrogen abstraction process to generate a thermodynamically more stable benzylic radical 2a-H. For the oxidation of aromatic C—H bond, the first step is the addition of benzoyloxy radical in A or D to the aromatic ring of mesitylene, which has a barrier of about 6 kcal mol-1 lower than that of the corresponding hydrogen abstraction. However, the newly generated cyclohexadienyl radical intermediate B or E is thermodynamically unfavorable by several kcal mol-1 due to the loss of aromaticity. Therefore, the barrier of the following hydrogen transfer becomes a critical factor in determining the reaction mode. The use of diradical A makes this process occur intramolecularly, and the entropic contribution (−TΔS) to the activation free energy can be ignored. This greatly decreases the barrier of the second step in the aromatic C—H functionalization, thereof leading to the exclusive aryl-selectivity in the diradical mediated reaction.

The present invention provides the phthaloyl peroxide (1) mediated hydroxylation of arenes and provides a new, selective method for the conversions of arenes to phenols. The hydroxylation procedure is performed under mild conditions without the utilization of metallic reagents or strong acids facilitating time, cost, and purification. Moreover, this methodology possesses broad functional group compatibility, has excellent selectivity for aromatic C—H bonds, and does not lead to overoxidation. The tolerance of the reaction toward a variety of functional groups permits the modification of advanced synthetic intermediates. Mechanistic insights into the reverse rebound process provide a novel strategy of selective C—H functionalization and lays the foundation for the discovery of new chemical transformations using diradicals.

General procedure for the hydroxylation of arenes: A borosilicate flask was equipped with a magnetic stir bar and neat or solid arene (0.2-0.8 mmol) was added. Addition of HFIP or TFE (2-5 mL) to provide a 0.2 M solution was followed by the addition of solid phthaloyl peroxide (1, 1.3 equiv.) in portions over 90 seconds. The reaction flask was placed in a heated oil bath (23-50° C.). After 3-24 hours, the reaction was removed from the oil bath and cooled to ambient temperature (23° C.). The reaction was then concentrated and under positive N2 pressure (to avoid potential air oxidation of the phenolic product) MeOH (3 mL) and saturated aqueous NaHCO₃ solution (0.2 mL) were added and the solution was stirred. After 12 hours, the reaction was quenched with pH 7.0 phosphate buffer (5 mL) and extracted with EtOAc (10 mL×3) and the combined organic layers were washed with brine (5 mL), dried over Na₂SO₄, and concentrated. The crude material was purified by silica gel column chromatography to afford the desired phenolic product.

Although the installation of oxygen into organic molecules is a fundamental process in organic synthesis, there is a lack of methods for the general, straightforward conversion of arenes to phenols. One of the major challenges in developing hydroxylation reactions of aromatic compounds is the products of the reactions are more reactive than the starting materials, leading to over-oxidation. Early hydroxylation reactions employing peroxides had limited success generating monohydroxylated products largely due to the restricted scope of the reactions. Two methods attempting to circumvent this problem using super acids to both activate the peroxides and subsequently deactivate the arenes were investigated. However, this approach also lacks generality and requires functionality that can tolerate exceptionally strong Brønsted acids. As a result of these shortcomings researchers frequently recourse to the use of Friedel-Crafts/Baeyer-Villiger sequences for the installation of oxygen into arenes.

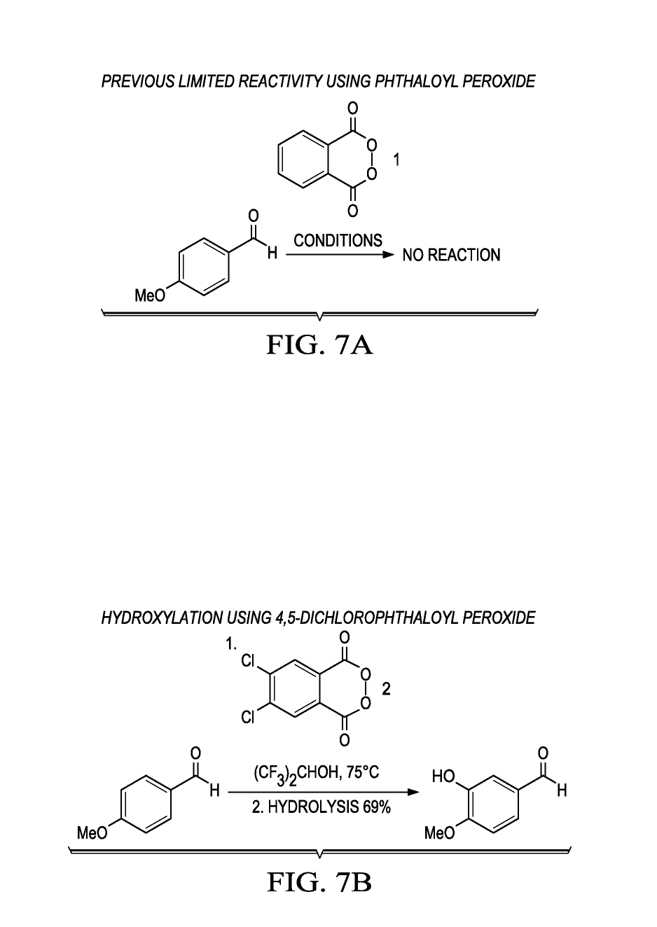

FIGS. 7A-7B are schematics of the reaction using phthaloyl peroxide (FIG. 7A) and 4,5-dichlorophthaloyl peroxide (FIG. 7B). Recently phthaloyl peroxide (1), a reagent studied in detail by Greene in the 1950's, was shown to monohydroxylate arenes while possessing a high degree of tolerance for a variety of functional groups. However, there are limitations to the arenes that are competent substrates, namely the arenes needed to be moderately electron rich. 4,5-dichlorophthaloyl peroxide (2), a reagent previously studied in the context of dihydroxylation reactions of styrenes and stillbenes, has improved reactivity when used in hexafluoroisopropanol (HFIP) at elevated temperatures. These conditions allow reactions with arenes that were previously unreactive toward phthaloyl peroxide (1) under the reported conditions. As a representative example, anisaldehyde failed to react with phthaloyl peroxide (1) under the previously reported conditions (FIG. 7A) while 4,5-dichlorophthaloyl peroxide (2) provided the hydroxylated product, isovanillin, in 69% yield as a single isomer (FIG. 7B).

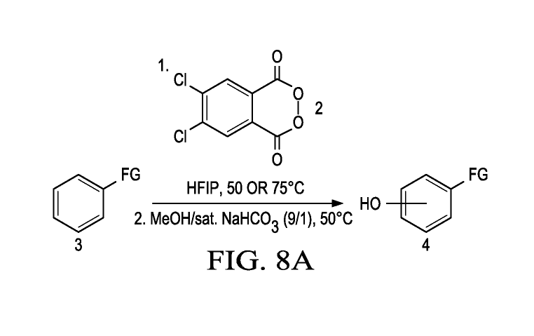

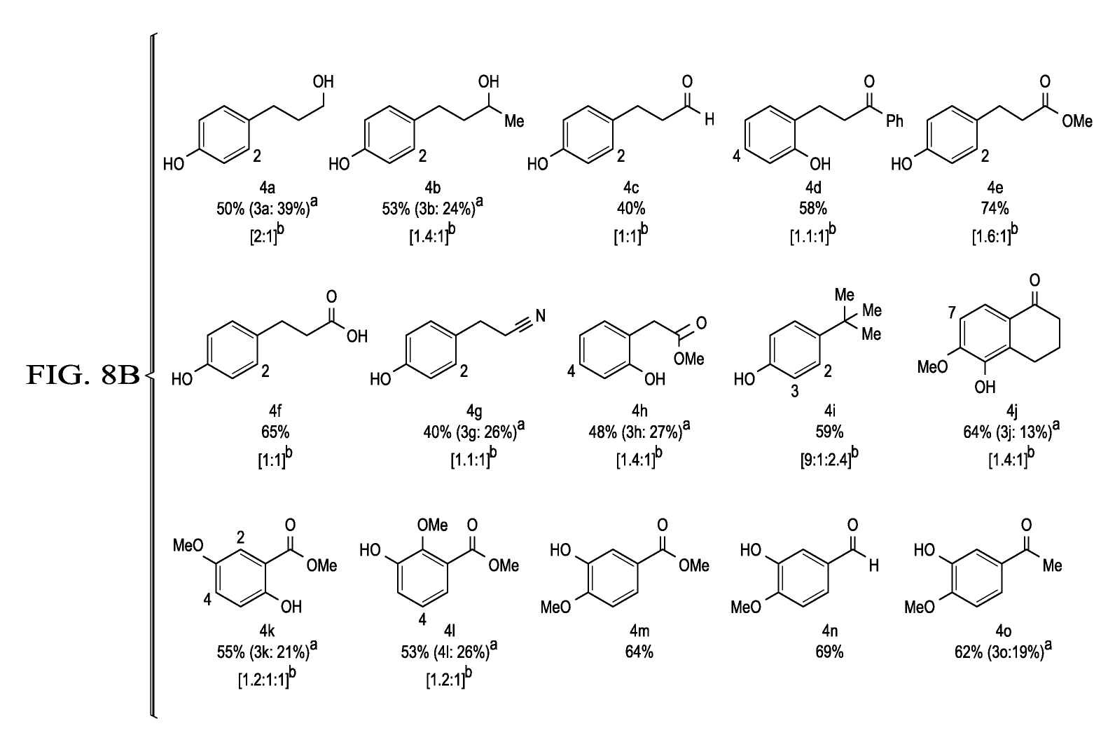

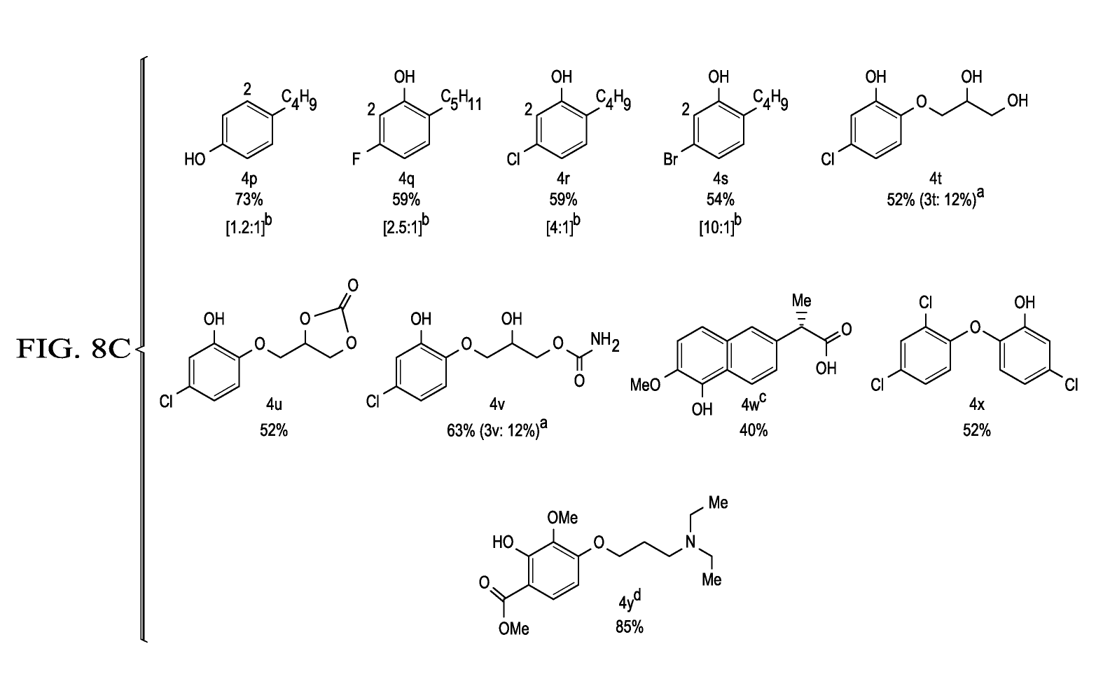

FIGS. 8A-8C are illustrations of the hydroxylation of arenes mediated by 4,5-dichlorophthaloyl peroxide. To examine the scope of the hydroxylation reaction mediated by 4,5-dichlorophthaloyl peroxide (2), two general sets of reaction conditions were developed. The oxidations are carried out using either 1.3 equivalents of 4,5-dichlorophthaloyl peroxide (2) at 50° C. or 2.5 equivalents heated to 75° C. Operationally the reaction proceeds without the need for special exclusion of air and the use of commercial grade HFIP is sufficient. Thermogravimetric analysis indicates that 4,5-dichlorophthaloyl peroxide has a point of decomposition at 135° C. Therefore, all reactions reported are conducted at or below 75° C. While we have not encountered exothermic reactions, appropriate precautions must be used similar to those for all experiments using peroxides. Isolated yields are given below each entry. The yield in parentheses refers to the starting material recovered. The minor regioisomeric positions are labeled with the respective carbon atom number and, after the major isomer, listed sequentially. Reaction conducted at 0° C. Prior to the addition of 4,5-dichlorophthaloyl peroxide (2) p-toluenesulfonic acid monohydrate (1.0 equiv.) was added to the solution of 3(y).

The products in FIGS. 8A-8C demonstrate the range of substituted arenes that can be successfully hydroxylated as well as functional groups that are tolerated, adding to those previously known. Primary and secondary alcohols are tolerated providing phenols (4a) and (4b). Similarly, a series of hydrocinnamyl derivatives with higher degrees of oxidation including aldehyde (3c), ketone (3d), ester (3e), carboxylic acid (3f), and nitrile (3 g) were transformed to the corresponding phenols. Removal of one methylene going from (3e) to (3h) led to a diminished yield of 48% for the oxidation generating ester (4h). For the first time a meta-hydroxylated product was generated by the reaction of tert-butyl benzene (3i) although the paraisomer was the major adduct. This product potentially arises through rearrangement before aromatization. Hydroxylation of 6-methoxy tetralone (3j) as well as methyl m-anisate (3k) and methyl o-anisate (3l) occurred in moderate yield and provided mixtures of isomeric products. Single regiosiomers were obtained in the hydroxylation of methyl p-anisate (3m), p-anisaldehyde (3n), and acetanisole (3o) driven by synergistic regiochemical directing effects of the methyl ether and carbonyls. Butyl benzene (3p) was converted in higher efficiency using 4,5-dichlorophthaloyl peroxide (2) in 73% compared to 49% conversion using phthaloyl peroxide (1). A series of halogenated alkyl benzene derivatives, (3q-s), were oxidized delineating the regioselectivities possible within these systems. As expected the halogens were not as strong as directing groups as alkyl substituents and within these substrates fluorine is a stronger director than chlorine which in turn is more effective than bromine.

As free alcohols were tolerated chlorphenesin glycol (3t) was hydroxylated providing (4t) as the sole regioisomer. The related chlorphenesin carbonate (3u) and carbamate (3v8) were similarly reacted providing the corresponding phenols 4u and 4v in 52% and 63% yield respectively. Hydroxylation of the free acid of naproxen (3w9) was achieved generating (4w) as the sole isomer isolated with the low yield, in part, due to air oxidation of the electron rich napthol (4w). The hydroxylation strategy also provided an alternative approach to access the antibacterial/antifugal agent triclosan (4x). 10 Hydroxylation of 2,4,4′-trichlorodiphenyl ether (3x) was achieved, regioselctively, forming triclosan (4x) in 52% yield. After testing additives it was found that amines (in their ammonium form) are tolerated. The addition of ptoluenesulfonic acid monohydrate (1.0 equiv.) prior to 4,5-dichlorophthaloyl peroxide (2) leads to the successful hydroxylation of the amine containing methyl vanillate derivative (3y) to generate the phenolic-amine (4y) in 85% yield.

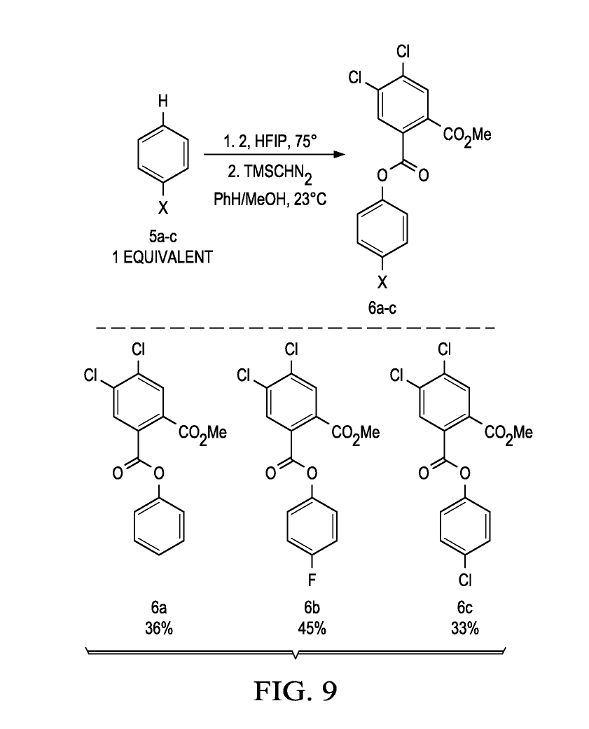

FIG. 9 is a schematic of the oxidation reaction of benzene, fluorobenzene, and chlorobenzene. Isolated yields for 1 equiv. of arene reacted with 2.5 equiv. 2 in HFIP at 75° C. The majority of previous arene oxidation procedures have focused on the hydroxylation of simple aromatics such as benzene, (typically used as solvent and substrate). However, to showcase the unique reactivity of our system benzene (5a), fluorobenzene (5b), and chlorobenzene (6c) were used and reacted with an excess, 2.5 equivalents of 4,5-dichlorophthaloyl peroxide (2), in HFIP at 75° C. As the phenolic products were volatile the intermediate mixed phthaloyl ester-acids were esterified and then fully characterized. While the yields were modest the reactivity of 4,5-dichlorophthaloyl peroxide (2) with these less reactive arenes is noteworthy as secondary oxidation of the products was found to not be competitive.

The hydrolysis of the intermediate phthaloyl ester-acid is typically conducted using a mixture of methanol and saturated aqueous sodium bicarbonate solution to expedite hydrolysis. However, the high reactivity of this class of esters has been noted and they have been dubbed “self immolative.” Exceedingly mild hydrolysis under neutral conditions and ambient temperature is possible.

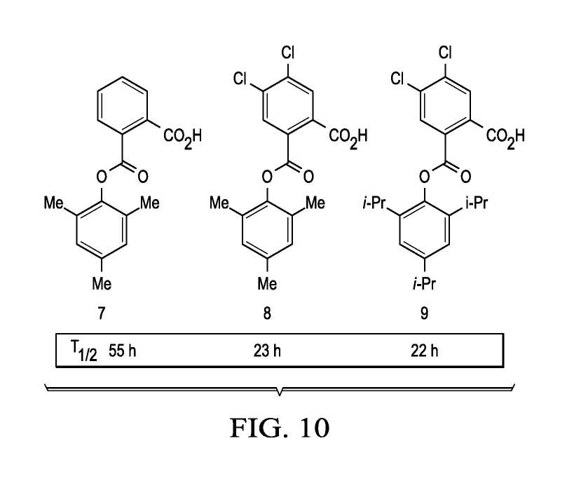

FIG. 10 is an image of the mixed phthaloyl ester-acids and their respective half-lives in pH 7.0 phosphate buffer/THF (1:1) solution at 23° C. Measurements of the half-lives of the intermediates under neutral conditions show that they undergo hydrolysis, with the 4,5-dichlorophthaloyl peroxide adducts cleaving more rapidly than phthaloyl peroxide adducts, as demonstrated by the hydrolysis of ester (7) versus ester (8). Importantly, the sterics of the ester are inconsequential as hydrolysis of ester (8) proceeds at essentially the same rate as ester (9).

Arene hydroxylation using 4,5-dichlorophthaloyl peroxide (2) provides a reliable method for the conversion of arenes to phenols. With enhanced reactivity, relative to the parent compound phthaloyl peroxide (1), this reagent can hydroxylate a wide range of arenes. In addition, a variety of functional groups including alcohols, diols, amines, carbamates, esters, aldehydes, ketones, and carboxylic acids are compatible, consistent with the hydroxylation reaction having potential broad applicability in synthesis.

The present invention provides a method of converting a substituted arene to a phenol. In general, the present invention includes an arene having 5, 6, 7, 8, 9, 10, 11, or 12 members in a single ring or a fused ring and optionally one or more substitutions on the arene that reacts with a 4,5-di-halogenated phthaloyl peroxide. For example, the arene may have the following structure:

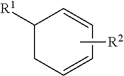

where R1 is a functional group that may be an alkyl group with one or more hydroxyl groups, a carbonyl group, an ester group, a methoxy, an ether group, or a nitrile. For example some of the compound including the R1 functional groups may be seen in FIG. 8. A non-limiting listing of some of the functional groups include:

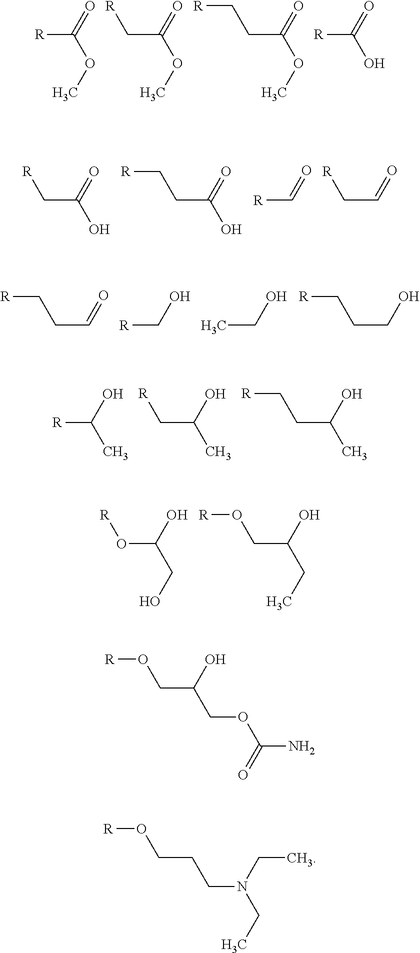

In addition, the arene includes one or more substitutions that may be hydroxly group, a methoxy group, or a halogen. The skilled artisan will understand that other functional groups may be used as well.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method for converting an aromatic hydrocarbon to a phenol comprising the steps of:
providing one or more aromatic hydrocarbons comprising one or more aromatic C—H bonds and one or more activated C—H bonds in a solvent;
adding a phthaloyl peroxide to the solvent;
reacting the one or more aromatic hydrocarbons and the phthaloyl peroxide to form one or more phenols, wherein one of the one or more aromatic C—H bonds is selectively oxidized in preference to one or more activated C—H bonds; and
purifying the one or more phenols.

2. The method of claim 1, wherein the one or more aromatic hydrocarbons comprise one or more functional groups.

3. The method of claim 1, wherein the one or more aromatic hydrocarbons comprise one or more functional groups selected from alkyl silanes, azides, allenes, nitriles, alkyl boronates, alcohols, halides, and epoxides.

4. The method of claim 1, wherein the one or more aromatic hydrocarbons comprise a 5, 6, 7, 8, 9, 10, 11, or 12 member ring or fused rings.

5. The method of claim 1, wherein the one or more aromatic hydrocarbons comprise one or more polycyclic aromatic hydrocarbons.

6. The method of claim 1, wherein the phthaloyl peroxide generates a di-radical that reacts by a reverse rebound mechanism.

7. The method of claim 1, wherein the solvent is trifluoroethanol or hexafluoroisopropanol.

8. The method of claim 1, further comprising the step of regenerating the phthaloyl peroxide.

9. A method of forming (+)-δ-tocopherol comprising the steps of:
providing dehydroxy-(+)-δ-tocopherol in a solvent;
adding a phthaloyl peroxide to the solvent;
reacting the dehydroxy-(+)-δ-tocopherol and phthaloyl peroxide to form a (+)-δ-tocopherol, wherein the phthaloyl peroxide generates a di-radical that reacts by a reverse rebound mechanism;
regenerating the phthaloyl peroxide; and
purifying the (+)-δ-tocopherol.

10. The method of claim 9, wherein the solvent is trifluoroethanol or hexafluoroisopropanol.

11. A method for converting an aromatic hydrocarbon to a phenol comprising the steps of:
providing one or more aromatic hydrocarbons comprising one or more aromatic C—H bonds and one or more activated C—H bonds in a solvent;
adding a phthaloyl peroxide to the solvent;
converting the phthaloyl peroxide to a di-radical;
contacting the di-radical with the one or more aromatic C—H bonds, wherein the phthaloyl peroxide generates a di-radical that reacts by a reverse rebound mechanism;
oxidizing selectively one of the one or more aromatic C—H bonds in preference to the one or more activated C—H bonds;
adding a hydroxyl group to the one of the one or more aromatic C—H bonds to form one or more phenols; and
purifying the one or more phenols.

12. The method of claim 11, wherein the aromatic hydrocarbon comprise one or more functional groups.

13. The method of claim 11, wherein the aromatic hydrocarbon comprise one or more functional groups selected from alkyl silanes, azides, allenes, nitriles, alkyl boronates, alcohols, halides, and epoxides.

14. The method of claim 11, wherein the aromatic hydrocarbons comprise a 5, 6, 7, 8, 9, or 10, 11, or 12 member ring or fused rings.

15. A method for generating a substituted arene to a phenol comprising the steps of:
providing one or more aromatic hydrocarbons comprising one or more functional groups in a solvent;
adding a halogenated phthaloyl peroxides to the solvent;
reacting the one or more aromatic hydrocarbons and the halogenated phthaloyl peroxides to hydroxylate the one or more aromatic hydrocarbons to form one or more substituted arenes comprising a hydroxyl group and the one or more functional groups; and
purifying the one or more substituted arenes.

16. The method of claim 15, wherein the halogenated phthaloyl peroxides is a poly-chloro phthaloyl peroxide, poly-bromo phthaloyl peroxide, poly-fluoro phthaloyl peroxide or a combination thereof.

17. The method of claim 15, wherein the one or more functional groups are selected from an alcohol group, a carbonyl group, an ester group, a methoxy, an ether group, a nitrile.

18. The method of claim 15, wherein the one or more aromatic hydrocarbons further includes one or more functional groups selected from an alcohol group, a carbonyl group, an ester group, a methoxy, an ether group, a nitrile.

19. The method of claim 15, wherein the one or more aromatic hydrocarbons is a 5, 6, 7, 8, 9, 10, 11, or 12 member ring or fused rings.

20. The method of claim 15, further comprising the step of regenerating the halogenated phthaloyl peroxides.
